(19) United States
(12) Patent Application Publication
(10) Pub. No.: US 2024/0409880 A1
(43) Pub. Date: Dec. 12, 2024

(54) METHODS FOR INCREASING ALGAE PRODUCTIVITY USING HIGH SILICA CONCENTRATIONS

(71) Applicant: Global Algae Technology, LLC, San Diego, CA (US)

(72) Inventors: David A. Hazlebeck, El Cajon, CA (US); Rodney Corpuz, Lihue, HI (US); Agnieszka Pinowska, San Diego, CA (US); Jesse Traller, San Diego, CA (US)

(21) Appl. No.: 18/741,502

(22) Filed: Jun. 12, 2024

Related U.S. Application Data
(60) Provisional application No. 63/507,730, filed on Jun. 12, 2023.

(51) Int. Cl. C12N 1/12; C12M 1/00

(52) U.S. Cl. CPC C12N 1/12; C12M 29/04

(57) ABSTRACT

The present disclosure describes cultivation methods for increasing productivity of algae strains by cultivating the algae in a high silica cultivation fluid. Also described are methods for using high silica concentrations to reduce contaminants in an algae culture. Also described are systems for supplementing cultivation fluid with silica.

### CROSS-REFERENCE TO RELATED APPLICATIONS

This application claims the benefit of and priority to U.S. Provisional Application No. 63/507,730 filed on Jun. 12, 2023 the content of which is incorporated by reference in its entirety.

### BACKGROUND

As worldwide petroleum deposits decrease, there is rising concern over petroleum shortages and the costs that are associated with the production of carbon-based fuel sources. Additionally, those concerned about the potential impact of global warming are seeking to explore methods to sequester CO₂ from the air and/or release more oxygen into the atmosphere.

Biofuel derived from algae is a possible alternative to petroleum-based fuels and provides substantial oxygen to the environment when productively grown. In particular, algae is known to be one of the most efficient plants for converting solar energy into cell growth, making it a good potential for a biofuel source. The use of algae as a biofuel source presents no exceptional problems, i.e., biofuel can be processed from algae as easily as from land-based plants. Also, algae can be grown heterotrophically or mixotrophically to produce materials for biofuel production. For example, microalgae can be grown on cellulosic and hemicellulosic sugars to produce lipids.

Processes involved in creating biofuel from plants are expensive relative to the process of extracting and refining petroleum. It is possible, however, that the cost of processing a plant-derived biofuel could be reduced by maximizing the rate of growth of the plant source.

Algae cultivation has also become widely recognized as a promising source of food, feed, biofuel, chemicals, polymers and nutraceuticals. Photosynthetic algae production offers the potential for an order of magnitude higher agricultural productivity than other plants. Some algae strains are preferred because of higher lipid content, better lipid profiles, or higher value compounds. Examples of some strains of commercial interest are Nitzschia for lipid content and profile; Haematococcus for astaxanthin content; Nannochloropsis and a variety of diatoms for EPA content; and Spirulina for phycocyanin and protein content.

While algae can efficiently transform solar energy into chemical energy via a high rate of cell growth, it has been difficult to create environments in which cell growth rates are optimized. Therefore, the economics of large-scale cultivation remains challenging. One way to improve the economics is through obtaining higher productivity in the cultivation systems, so numerous approaches have been taken to improve the productivity of various cultivation systems.

In light of the above, it is an object of the present disclosure to provide a method for maximizing the growth and lipid content of algae cultures.

### SUMMARY

In a first aspect, a method for cultivating an algae strain in an aqueous culture is provided, the method comprising growing the algae strain in a cultivation fluid comprising a silica concentration of higher than about 500 μM. The cultivation fluid may comprise a silica concentration of at least about 2 mM. The cultivation fluid may comprise a silica concentration of at least about 2.5 mM. The cultivation fluid may comprise a silica concentration of at least about 5 mM.

The aqueous culture may comprise a silica loading between about 2 mM and 15 mM. The silica loading may be higher than 5 mM during a portion of a cultivation period, and the method may further comprise reducing the silica loading to between about 0.5 mM and about 5 mM for at least one day prior to harvesting.

In embodiments, the algae strain is a diatom. In embodiments, the algae strain is not a diatom.

The silica concentration may be higher than about 500 μM at a beginning of the cultivation period.

The method may further comprise: harvesting the algae strain to produce a harvest; and adding recycled media from the harvest to a new aqueous culture.

The method may further comprise at least one of: (a) adding diatomaceous earth to at least a portion of the recycled media before adding the recycled media to the new aqueous culture; and (b) extracting silica cell walls from the harvest; and adding the silica cell walls to the recycled media. In embodiments, the method further comprises heating the recycled media to between about 40° C. and about 100° C. The method further comprises compressing, pressurizing, and heating the recycled media to between about 100° C. and about 350° C.

The recycled media may have a pH greater than 9.5. The recycled media may have a pH greater than 10. The recycled media may have a pH greater than 10.5.

The method may further comprise filtering the recycled media after adding the diatomaceous earth and before adding the recycled media to the new aqueous culture.

In another aspect, a method for reducing contaminants in an aqueous algae culture is provided herein, the method comprising increasing a silica concentration of the culture to higher than about 1 mM. The method may further comprise reducing the silica concentration to between about 500 μM and about 1 mM silica at about one to about four days after increasing the silica concentration.

In embodiments, the method may comprise increasing the silica concentration to higher than about 10 mM. The method may further comprise reducing the silica concentration to between about 500 μM and about 10 mM silica at about one to about four days after increasing the silica concentration to higher than about 10 mM. The silica concentration may be reduced to between about 2 mM and about 5 mM.

The method may comprise cultivating the culture at a silica concentration of more than about 500 μM silica and less than about 10 mM silica prior to increasing the silica concentration.

In embodiments, the algae culture comprises an algae strain that does not require silica for growth. In embodiments, the algae culture comprises an algae strain that requires silica for growth.

In another aspect, an algae cultivation system is provided, the system comprising: an algae cultivation unit configured to produce an algae slurry in a cultivation fluid; a harvest unit configured to produce a concentrated algae slurry and a recycled cultivation fluid from the algae slurry; and a diatomaceous earth unit configured to dissolve diatomaceous earth into at least a portion of the recycled cultivation fluid.

In embodiments, the cultivation system further comprises a slurry conduit configured to transfer the algae slurry from the algae cultivation unit to the harvest unit; a recycled media conduit configured to transfer at least a portion of the recycled cultivation fluid from the harvest unit to the diatomaceous earth unit; and a diatomaceous earth conduit configured to transfer recycled cultivation fluid from the diatomaceous earth unit to the algae cultivation unit.

The cultivation system may further comprise a filter configured to filter the recycled cultivation fluid from the diatomaceous earth unit. The cultivation system may further comprise: a slurry conduit configured to transfer the algae slurry from the algae cultivation unit to the harvest unit; a recycled media conduit configured to transfer at least a portion of the recycled cultivation fluid from the harvest unit to the diatomaceous earth unit; a diatomaceous earth conduit configured to transfer recycled cultivation fluid from the diatomaceous earth unit to the filter; and a filtered media conduit configured to transfer the recycled cultivation fluid from the filter to the algae cultivation unit. The cultivation system may further comprise an algae processing unit configured to extract at least a portion of silica cell walls from the concentrated algae slurry. The cultivation system may further comprise a silica cell wall conduit configured to transfer the silica cell walls from the algae processing unit to the diatomaceous earth unit.

In another aspect, provided herein is a method for cultivating an algae strain using any of the cultivation systems described herein.

### BRIEF DESCRIPTION OF THE DRAWINGS

Non-limiting embodiments of the present invention will be described by way of example with reference to the accompanying figures, which are schematic and are not intended to be drawn to scale. In the figures, each identical or nearly identical component illustrated is typically represented by a single numeral. For purposes of clarity, not every component is labeled in every figure, nor is every component of each embodiment of the invention shown where illustration is not necessary to allow those of ordinary skill in the art to understand the invention.

FIG. 1 is a schematic illustration of an algae cultivation system in accordance with some embodiments of the present disclosure.

FIG. 2 is a schematic illustration of an algae cultivation system in accordance with some embodiments of the present disclosure.

### DETAILED DESCRIPTION

The present invention has been described in terms of one or more preferred embodiments, and it should be appreciated that many equivalents, alternatives, variations, and modifications, aside from those expressly stated, are possible and within the scope of the invention.

The process of using algae for biofuel, food sources, CO₂ sequestration, etc., necessarily begins by growing the algae cells. There are numerous expenses associated with growing algae, including the costs of nutrients for feeding the algae, and the costs of adding energy, e.g. via artificial light sources, into the growth systems. Growing algae is most efficient in a controlled environment where a nutrient mixture is provided in the algae growth media. Growing in outdoor ponds using sunlight helps reduce the costs of energy input needed, but this solution exposes the algae to contaminants in the outdoor environment. Adjusting the nutrient growth mixtures is one way to simultaneously maximize algal cell growth and reduce contaminants.

Diatoms are single-celled algae surrounded by a cell wall composed primarily of silica. During growth, silicate metabolism is linked to cell growth and division. Therefore, silica is regarded as an essential macronutrient for diatomaceous algae growth, and can be a limiting nutrient if not available in amounts that support growth. While non-diatomic algae are typically grown in no more than 200 μM silica concentration, typical diatom media includes up to 500 μM silica concentration (Andersen, R.A., Ed. (2005) Algal Culturing Techniques. Elsevier Academic Press, pgs 429-538).

Additionally, due to their siliceous wall, diatoms can tolerate a higher amount of silica in their environment than most other organisms. Therefore, high silica concentrations can mitigate the growth of certain green algae, plants, and bacterial species. As some bacterial species can accelerate dissolution of silica in diatoms, preventing bacterial growth can further enhance diatom productivity.

While cultivating diatoms at high silica concentrations, the inventors noticed that the high silica media was able to kill or reduce the growth of other non-diatom algae types. For these, and other reasons, the inventors developed methods of cultivating algae using high silica media/cultivation fluid. The disclosed methods increase productivity of algae strains, reduce contaminants in algae cultures, and increase the lipid content of cultivated algae.

In a first aspect, a method for cultivating an algae strain in an aqueous culture is provided, the method comprising growing the algae strain in a cultivation fluid comprising a silica concentration of higher than about 500 μM. The silica concentration may be at least about 2 mM, 2.5 mM, or 5 mM. The silica concentration may be between 500 μM and 20 mM, or any concentration or range in between, e.g., about 750 μM, about 1 mM, about 1.5 mM, about 2 mM, about 2.5 mM, about 3 mM, about 4 mM, about 5 mM, about 6 mM, about 7 mM, about 8 mM, about 9 mM, about 10 mM, about 11 mM, about 12 mM, about 13 mM, about 14 mM, about 15mM, about 20 mM, etc. The algae strain may be a diatom. The algae strain may be a non-diatom, wherein the strain is cultivated in a silica concentration that is not toxic to the strain.

The method may comprise growing the algae strain in a cultivation fluid comprising between about 10 mM and about 20 mM silica in order to mitigate growth of contaminants in the algae culture.

The silica loading of the culture, i.e. silica in the cultivation fluid and incorporated into the algae cell wall, may be between about 2 mM and about 14 mM, or any range or concentration in between. The silica loading may be higher than 5 mM, e.g. about 6 mM, about 7 mM, about 8 mM, about 9 mM, about 10 mM, about 11 mM, about 12 mM, about 13 mM, about 14 mM, about 15 mM, about 20 mM, etc.

The silica loading may be greater than 5 mM during a portion of a cultivation period, then reduced to between about 500 μm and about 5 mM or any concentration or range in between, e.g. about 750 μM, about 1 mM, about 1.5 mM, about 2 mM, about 2.5 mM, about 3 mM, about 4 mM, about 5 mM for at least one day prior to harvesting. This is done to increase the lipid content, reduce the cost of silica supply, and reduce the silica content in the harvested algae biomass.

The algae may be cultivated in cultivation fluid comprising an initial concentration of higher than about 500 μM silica.

Cultivation fluid comprising a high silica concentration can be expensive. Therefore, in embodiments, after the algae is harvested, the remaining cultivation fluid may be recycled (e.g. recycled media) for use in cultivation of a new culture. Any remaining silica in the recycled cultivation fluid will increase the silica concentration in the new culture.

The recycled cultivation fluid may have a pH of greater than 9.5, e.g. about 9.6, about 9.8, about 10.0, about 10.2, about 10.4, about 10.6, about 10.8, about 11, etc.

At least a portion of the recycled cultivation fluid having a pH greater than 9.5 may be supplemented with one or both of diatomaceous earth and silica cell walls extracted from the harvested algae before it is used for further cultivation. Silica in the diatomaceous earth will dissolve into the recycled cultivation fluid and increase the silica concentration in the culture. In embodiments at least a portion of the diatomaceous earth is recycled silica cell walls from the harvested algae. In embodiments, the method further comprises extracting the silica cell walls from the harvested algae. After adding diatomaceous earth, the temperature of the recycled media may be raised to above ambient temperature, e.g. to about 50° C., about 60° C., about 90° C., about 100° C., etc. to increase the rate of dissolution. The recycled media may be compressed, pressurized, and heated to between about 100° C. and about 350° C., or any temperature or range in between, e.g. 110° C., 120° C., 140° C., 160° C., 180° C., 200° C., 250° C., etc. After supplementing the recycled cultivation fluid with diatomaceous earth, the recycled cultivation fluid may be filtered before it is used for further cultivation. In embodiments, the method comprises extracting silica cell walls from the harvested algae and is added to the recycled media alone, without the addition of diatomaceous earth. After adding the silica cell walls, the temperature of the recycled media may be raised to above ambient temperature, e.g. to about 50° C., about 60° C., about 90° C., about 100° C., etc. to increase the rate of dissolution. The recycled media may be compressed, pressurized, and heated to between about 100° C. and about 350° C., or any temperature or range in between, e.g. 110° C., 120° C., 140° C., 160° C., 180° C., 200° C., 250° C., etc.

Higher silica concentrations in algae cultures are helpful in efficiently reducing contaminants and may therefore be used continuously or temporarily during algae cultivation. Accordingly, in a second aspect, provided herein is a method for reducing contaminants in an aqueous algae culture, comprising increasing a silica concentration to the culture to higher than about 1 mM. The method may comprise increasing the silica concentration to higher than about 10 mM. The concentration may be brought to above 10 mM for a short period of time, e.g. one to four days, for example about 1 day, about 2 days, about 3 days, about 4 days, and any time or range in between. The method may further comprise cultivating the algae culture at a concentration of more than about 500 μM silica and less than about 10 mM silica before and after raising the concentration to above 10 mM. By raising the silica concentration above 10 mM for only 1 to 4 days, cultivation costs may be mitigated. Raising the silica concentration to above 10 mM may be done in regular intervals as a maintenance procedure. Additionally, or alternatively, the culture may be monitored for levels of contaminants, and the silica concentration may be raised when the contaminant levels are higher than desirable. In embodiments, the algae culture comprises an algae strain that does not require silica for growth such as Spirulina, Chlorella, Nanochloropsis, etc. In other embodiments, the algae culture comprises an algae strain that requires silica for growth such as Nitzschia, Cyclotella, Chaetoceros, Thalassiosira, and other diatoms.

In a third aspect, provided herein is a method for cultivating an algae strain in an aqueous culture, the method comprising growing the algae strain in a cultivation fluid, wherein the silica loading in the culture comprises a silica loading higher than 5 mM during a portion of a cultivation period, and wherein the silica loading is reduced to between about 0.5 mM and about 5 mM for at least one day prior to harvesting. The method may further comprise growing the algae strain in a cultivation fluid wherein the silica loading is higher than 10 mM during a portion of the cultivation period.

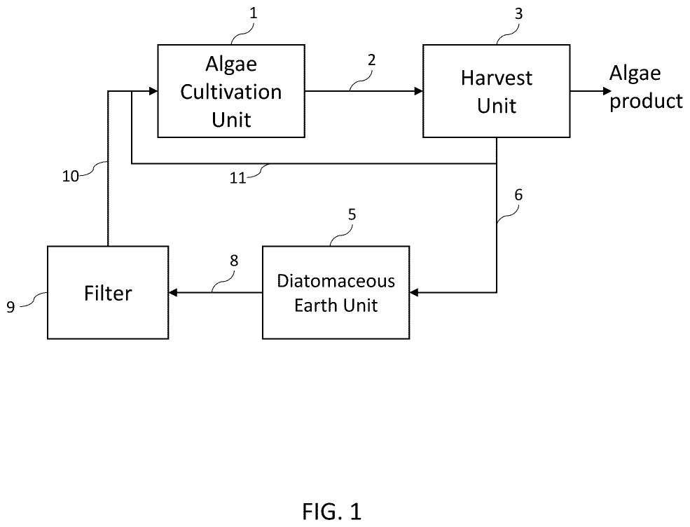

In a fourth aspect, and as illustrated in FIG. 1, provided herein is an algae cultivation system comprising an algae cultivation unit (1) configured to produce a algae slurry in a cultivation fluid; a harvest unit (3) configured to produce a concentrated algae slurry and a recycled cultivation fluid from the algae slurry; a diatomaceous earth unit (5) configured to dissolve diatomaceous earth into at least a portion of the recycled cultivation fluid. The algae cultivation system may further comprise a filter (9) configured to separate diatomaceous earth from the recycled cultivation fluid and generate a filtered recycled media. Examples of suitable harvest systems are described in U.S. Pat. Nos. 10,501,721 B2, 10,351,815 B2, and U.S. Patent Publication No. US2020/0299636A1, each of which are incorporated by reference, in their entirety and for all purposes, herein.

The cultivation system may further comprise: a slurry conduit (2) configured to transfer the cultivation algae slurry from the algae cultivation unit (1) to the harvest system (3); a first recycled media conduit (6) configured to transfer at least a portion of the recycled cultivation fluid from the harvest unit (3) to the diatomaceous earth unit (5); a diatomaceous earth conduit (8) configured to transfer the recycled cultivation fluid from the diatomaceous earth unit (5) to the algae cultivation unit (1). In embodiments in which the system comprises a filter (9), the diatomaceous earth conduit (8) transfers the recycled cultivation fluid from the diatomaceous earth unit (5) to the filter (9); and the system further comprises a filtered media conduit (10) configured to transfer the recycled cultivation fluid from the filter (9) to the algae cultivation unit (1). The system may further comprise a second recycled media conduit (11) configured to transfer a portion of the recycled media from the harvest unit (3) to the cultivation unit (1).

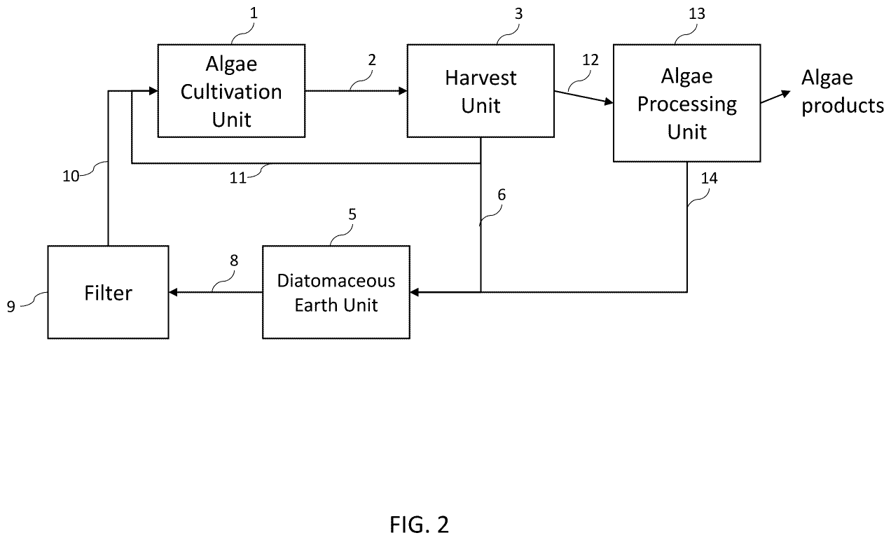

In an embodiment, and as illustrated in FIG. 2, the algae cultivation system further comprises an algae processing unit (13) configured to extract at least a portion of the silica cell walls from the algae concentrated algae slurry, and a silica cell wall conduit (14) configured to transfer the extracted silica cell walls from the algae processing unit (13) to the diatomaceous earth unit (5). The algae processing unit (13) may be additionally configured to extract other components such as proteins, lipids, carbohydrates, nucleic acids, pigments, etc. before or after extracting the silica cell walls.

The cultivation systems described herein may be used to cultivate algae and maintain a

high concentration of silica in the cultivation fluid. Accordingly, in an embodiment, a method for cultivating an algae strain having a silica cell wall in a first aqueous culture is provided, the method comprising: growing the algae strain in a cultivation system, such as a cultivation system described herein; harvesting a concentrated algae slurry and separating a recycled cultivation fluid from the algae slurry; dissolving diatomaceous earth into at least a portion of the recycled cultivating fluid; and adding the recycled cultivation fluid comprising the dissolved diatomaceous earth to a second aqueous culture. The method may further comprise processing the concentrated algae slurry to separate the silica cell walls from at least a portion of the algae in the concentrated algae slurry; and adding the silica cell walls to the recycled cultivation fluid comprising the dissolved diatomaceous earth.

The terms “algae culture”, “aqueous culture”, “aqueous algae culture”, and “slurry” are

used interchangeably and refer to an algae growth in an aqueous environment such as water or a medium. The algae culture is grown in a container that retains liquid, such as a pond, a tank, or a raceway.

A “cultivation fluid” is an aqueous medium comprising nutrients that promote algae growth.

A “cultivation period” refers to a period of time during which an algae culture is grown.

“Diatomaceous earth” is a soft, siliceous sedimentary rock comprised of fossilized remains of diatoms or cell walls of diatoms. The cell walls may be recycled from processing of the algae biomass and extracting of cell wall material.

“Silica loading” is the combined silica content in the algae cell walls and silica in solution in the algae culture.

“Harvesting” algae refers to the process of collecting or removing algae that has grown from an algae culture.

“Silica concentration” is silica in a soluble form in aqueous solution such as the algae culture, cultivation medium, cultivation fluid, or recycled media.

### Miscellaneous

Unless otherwise specified or indicated by context, the terms “a”, “an”, and “the” mean “one or more.” For example, “a molecule” should be interpreted to mean “one or more molecules.”

As used herein, “about”, “approximately,” “substantially,” and “significantly” will be understood by persons of ordinary skill in the art and will vary to some extent on the context in which they are used. If there are uses of the term which are not clear to persons of ordinary skill in the art given the context in which it is used, “about” and “approximately” will mean plus or minus ≤10% of the particular term and “substantially” and “significantly” will mean plus or minus >10% of the particular term.

As used herein, the terms “include” and “including” have the same meaning as the terms “comprise” and “comprising.” The terms “comprise” and “comprising” should be interpreted as being “open” transitional terms that permit the inclusion of additional components further to those components recited in the claims. The terms “consist” and “consisting of” should be interpreted as being “closed” transitional terms that do not permit the inclusion additional components other than the components recited in the claims. The term “consisting essentially of” should be interpreted to be partially closed and allowing the inclusion only of additional components that do not fundamentally alter the nature of the claimed subject matter.

All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Recitation of ranges of values herein are merely intended to serve as a shorthand method of referring individually to each separate value falling within the range, unless otherwise indicated herein, and each separate value is incorporated into the specification as if it were individually recited herein. For example, if a concentration range is stated as 1% to 50%, it is intended that values such as 2% to 40%, 10% to 30%, or 1% to 3%, etc., are expressly enumerated in this specification. These are only examples of what is specifically intended, and all possible combinations of numerical values between and including the lowest value and the highest value enumerated are to be considered to be expressly stated in this disclosure. Use of the word “about” to describe a particular recited amount or range of amounts is meant to indicate that values very near to the recited amount are included in that amount, such as values that could or naturally would be accounted for due to manufacturing tolerances, instrument and human error in forming measurements, and the like. All percentages referring to amounts are by weight unless indicated otherwise.

In those instances where a convention analogous to “at least one of A, B and C, etc.” is used, in general such a construction is intended in the sense of one having ordinary skill in the art would understand the convention (e.g., “a system having at least one of A, B and C” would include but not be limited to systems that have A alone, B alone, C alone, A and B together, A and C together, B and C together, and/or A, B, and C together.). It will be further understood by those within the art that virtually any disjunctive word and/or phrase presenting two or more alternative terms, whether in the description or figures, should be understood to contemplate the possibilities of including one of the terms, either of the terms, or both terms. For example, the phrase “A or B” will be understood to include the possibilities of “A” or ‘B or “A and B.”

All references, including publications, patent applications, and patents, cited herein are hereby incorporated by reference to the same extent as if each reference were individually and specifically indicated to be incorporated by reference and were set forth in its entirety herein.

Preferred aspects of this invention are described herein, including the best mode known to the inventors for carrying out the invention. Variations of those preferred aspects may become apparent to those of ordinary skill in the art upon reading the foregoing description. The inventors expect a person having ordinary skill in the art to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

### EXAMPLES

### Example 1

Nitzschia inconspicua was cultivated at pH 9.6 with a silica loading of 10-11 mM in an open raceway pond for 20 days. Silica was added to the culture at a concentration of 1-3 mM each day to maintain the 10-11 mM silica loading as the algae grew and the batch volume increased. The silica loading was reduced to below 5 mM by diluting the culture without adding silica. The third day after reducing the silica loading, the loading was 2 mM. The algae slurry was harvested on the third day and analyzed for lipid content. The lipid content was 47% of the ash-free dry weight of the biomass. This is a substantial increase in lipid content compared to the typical 5-10% lipid content when the algae is grown in standard media with silica concentrations below 500 μM.

### Example 2

Algae was cultivated at pH 10.4, then harvested to separate the culture into an algae slurry and recycled media. The silica concentration in the recycled media was below the detection limit, <10 μM. Diatomaceous earth was added to a portion of the recycled media and the mixture was heated to 60° C. In less than a day, the concentration of dissolved silica in the recycled media was 5 greater than 10 mM.

1. A method for cultivating an algae strain in an aqueous culture, the method comprising growing the algae strain in a cultivation fluid comprising a silica concentration of higher than about 500 μM.

2. The method of claim 1, wherein the cultivation fluid comprises a silica concentration of at least about 2 mM.

3. The method of claim 1, wherein the culture comprises a silica loading between about 2 mM and 15 mM.

4. The method of claim 1, wherein the culture comprises a silica loading higher than 5 mM during a portion of a cultivation period, and wherein the method further comprises reducing the silica loading to between about 0.5 mM and about 5 mM for at least one day prior to harvesting.

5. The method of claim 1, wherein the silica concentration is higher than about 500 μM at a beginning of the cultivation period.

6. The method of claim 1, further comprising: harvesting the algae strain to produce a harvest; and adding recycled media from the harvest to a new aqueous culture.

7. The method of claim 6, further comprising adding diatomaceous earth to at least a portion of the recycled media before adding the recycled media to the new aqueous culture.

8. The method of claim 7, further comprising heating the recycled media to between about 40° C. and about 100° C.

9. The method of claim 6, further comprising compressing, pressurizing, and heating the recycled media to between about 100° C. and about 350° C.

10. The method of claim 6, further comprising extracting silica cell walls from the harvest; and adding the silica cell walls to the recycled media.

11. The method of claim 6, wherein the recycled media has a pH greater than 9.5.

12. A method for reducing contaminants in an aqueous algae culture, the method comprising increasing a silica concentration of the culture to higher than about 1 mM.

13. The method of claim 12, further comprising reducing the silica concentration to between about 500 μM and about 1 mM silica at about one to about four days after increasing the silica concentration.

14. The method of claim 12, comprising increasing the silica concentration to higher than about 10 mM.

15. The method of claim 14, further comprising reducing the silica concentration to between about 500 μM and about 10 mM silica at about one to about four days after increasing the silica concentration.

16. The method of claim 14, wherein prior to increasing the silica concentration, the method comprises cultivating the culture at a silica concentration of more than about 500 μM silica and less than about 10 mM silica.

17. An algae cultivation system comprising:
an algae cultivation unit configured to produce an algae slurry in a cultivation fluid;
a harvest unit configured to produce a concentrated algae slurry and a recycled cultivation fluid from the algae slurry;
and a diatomaceous earth unit configured to dissolve diatomaceous earth into at least a portion of the recycled cultivation fluid.

18. The cultivation system of claim 17 further comprising: a filter configured to filter the recycled cultivation fluid from the diatomaceous earth unit.

19. The cultivation system of claim 17, further comprising an algae processing unit configured to extract at least a portion of silica cell walls from the concentrated algae slurry.

20. The cultivation system of claim 19, further comprising:
a silica cell wall conduit configured to transfer the silica cell walls from the algae processing unit to the diatomaceous earth unit.
